(19) United States
(12) Patent Application Publication
(10) Pub. No.: US 2024/0122737 A1
(43) Pub. Date: Apr. 18, 2024

(54) Catheter System

(71) Applicant: Leonid Shendelman, Brooklyn, NY (US)

(72) Inventor: Leonid Shendelman, Brooklyn, NY (US)

(21) Appl. No.: 18/226,238

(22) Filed: Jul. 25, 2023

Related U.S. Application Data
(63) Continuation-in-part of application No. 16/555,111, filed on Aug. 29, 2019, now Pat. No. 11,707,602.

(51) Int. Cl. A61F 5/44; A61M 25/00

(52) U.S. Cl. CPC A61F 5/4408; A61M 25/0017; A61M 25/10

(57) ABSTRACT

An apparatus for supporting a urinary catheter collection reservoir on an article of footwear or clothing of a catheter user. A catch is fastened to the article of footwear or clothing. A carrier includes a hook portion configured to be received within and supported by the catch. The carrier also includes a cradle portion configured to receive and support at least a portion of said reservoir.

A urine reservoir constructed to wrap around and attach to a catheter user's lower leg above the ankle so that the weight of the reservoir is supported on the ankle and above the foot. The reservoir is formed from overlapping, elongated strips of flexible, water-impermeable material having a central portion and opposed end portions. An internal urine cavity is formed in the central portion between the strips. First and second inter-engageable fasteners are formed at opposed, lengthwise ends of said strips to connect the reservoir around user's lower leg.

### CROSS REFERENCE TO RELATED APPLICATIONS

This is a continuation-in-part application of U.S. Ser. No. 16/555,111 entitled Catheter System, filed Aug. 29, 2019, which is incorporated herein by reference in its entirety.

### FIELD OF THE INVENTION

The present invention relates to catheter systems. More particularly, the present invention relates to catheter systems that are more comfortable to wear and easier to maintain.

### BACKGROUND OF THE INVENTION

People dealing with urinary incontinence often require, for either short term (up to two weeks or less) usage or long term (greater than two weeks up to about three months or more) usage, an indwelling catheter, typically a Foley catheter, connected to an exterior urine collection and drainage bag or reservoir. Mobile and active people, male or female, who have to wear such a reservoir on their bodies, are faced with a number of challenges, such as undesired reservoir and/or catheter movement, urine leakage, discomfort during insertion of the catheter through the person's urethra and into the bladder, irritation and soreness of the urethra and the bladder especially over long term use, undesired enlargement of and potential damage to the urethral passage, wardrobe issues, and difficulty of urine drainage from the reservoir, just to name a few such challenges.

The known reservoir is customarily attached to the person's thigh or upper leg, by one or two straps, and is connected to the indwelling catheter by a plastic, hollow, flexible tube through which the urine flows from the catheter to the reservoir. As urine collects in the reservoir, its increased weight may pull on the catheter. For example, a reservoir filled with about 500 milliliters (about 1 pint) of urine weighs about half a kilogram (about 1 pound). Such a weighted reservoir may cause discomfort and irritation to the delicate tissue of the urethra and bladder especially over long term usage, and may also pull the catheter down and cause the connections of the tube to the catheter and the reservoir to loosen and disengage, thereby causing discomfort, urine leaking, soiling, and embarrassment, etc. These problems are aggravated when the person is active and mobile during such daily activities as walking, running, driving, other physical work, etc.

Further problems relate to emptying the urine from many prior art reservoirs, as well as attaching the reservoir to the thigh or upper leg. Either the person must undress in order to empty the reservoir, or the person must wear loose-fitting pants to allow the person to roll up the pant leg far enough to gain access to the reservoir. Both methods are cumbersome and not always comfortable. Moreover, if a man must drain the reservoir in a public restroom, then he is faced with an embarrassing situation.

Another disadvantage is that there is insufficient support for many prior art reservoirs beyond the straps fitted around the thigh or upper leg. The straps are both tight and uncomfortable, yet insecure, i.e., the straps tend to restrict blood circulation, yet could not always prevent the reservoir from eventually slipping down the leg. Also, many art reservoirs prevent the person from wearing close-fitting street clothes, because the reservoir outline could show through the clothes, and because the pants leg could not be rolled up high enough to allow the person to gain ready access to the reservoir.

The drain on many prior art reservoirs also presents problems to some people. Individuals with shaky or arthritic hands or with neurologic pathologies, or some elderly, young, or handicapped, individuals, often have difficulty removing a plug, or screwing a cap off, or turning a valve, to open the drain, as well as with the reverse procedure to close the drain. Some people also may soil their hands or on adjacent body areas while draining the reservoir. These problems with many prior art catheters may be so great that some people may have difficulty in coping with them. Hence, in those instances, the only alternative is to wear bulky and embarrassing diapers, or to refuse to go out in public at all. For all these reasons, the urine-incontinent man or woman is often severely handicapped in both his economic and social life. Therefore, it would be desirable to provide a catheter system that is simple to use, and that alleviates one or more of the above-identified problems.

### SUMMARY OF THE INVENTION

The present invention relates to an apparatus for securely holding a urinary catheter reservoir in a fixed position proximate a catheter user's ankle. The apparatus securely and stably holds the reservoir in place, even during regular day-today or in hospitals or other medical settings, where users may need to remain connected to the catheter for an extended period of time. Additionally, the apparatus helps prevent accidental disconnection or displacement of the catheter, which in turn reduces the risk of infections and other complications associated with catheter use. Overall, the apparatus provides a reliable and effective solution for holding urinary catheter reservoirs in place, thereby improving patient comfort and safety in a wide range of environments.

In one exemplary, preferred embodiment, the invention comprises an apparatus for supporting a urinary catheter collection reservoir, which is worn outside of the human body rather than attached or placed on it. The apparatus supports the catheter on an article of footwear or clothing of a catheter user. The apparatus generally comprises a catch having means for fastening the catch to the article, and a carrier including a hook portion configured to be received within and supported by the catch, and a cradle portion configured to receive and support at least a portion of the reservoir. Preferably, the catch and carrier are configured and sized so that the entire weight of the reservoir is supported on the article and not on the user's foot or ankle.

In one preferred embodiment, the catch comprises an elongate strip of material, and the fastening means are located at each end of the strip. In this embodiment, the strip forms a pocket between the article and the strip when fastened to the article, and the pocket has a size and shape that compliments the size and shape of the hook portion of the carrier.

In other preferred embodiments, the catch may include a pocket having a shape and size that compliments the shape and size of the hook portion of the carrier. In other preferred embodiments, the catch comprises a two-ply strip of material, wherein the pocket is formed between the plies of material.

In one preferred embodiment, the fastening means comprises one or more mechanical fasteners selected from the group consisting essentially of rivets, pins, Velcro tabs, and snaps. The fastening means may also comprise an adhesive.

In one preferred embodiment, the carrier comprises an S-shaped strip of rigid material, having a mounting-hook portion, and a U-shaped cradle portion conjoined by a central shank portion.

In another preferred embodiment, the invention comprises a urinary catheter system generally comprising an indwelling catheter, a urine reservoir, and a flexible tube connecting the catheter to the urine reservoir. In one preferred embodiment, the indwelling, catheter has a tip inserted into a person's bladder. The tip has an opening through which urine from the bladder flows into the catheter.

The urine reservoir is constructed to wrap around and attach to a catheter user's lower leg above the ankle. The reservoir preferably includes inner and outer overlapping, elongated strips of flexible, water-impermeable material having a central portion and opposed end portions. The strips have a longitudinal length and a transverse width. An internal urine cavity is formed in the central portion between the strips by a sealed periphery comprising upper and lower longitudinal seams and left and right lateral seams.

First and second inter-engageable fasteners are formed at opposed, lengthwise ends of the strips. In preferred embodiments, the urine reservoir has a length sufficient to connect end-to-end at the said fasteners, and to completely surround a user's lower leg. In other embodiments, the reservoir has multiple fasteners arranged at different lengthwise positions to change the connected circumference of the urine reservoir. In one preferred embodiment, the fasteners comprise male tabs formed at one end and complimentarily-shaped and sized female slits formed at the other end.

In one preferred embodiment, the walls of the urine reservoir are corrugated in the central portion forming the urine cavity. Preferably, the corrugated walls of the urine reservoir have lengthwise-extending pleats so that the reservoir can expand and contract in the widthwise direction or vertical direction relative to the leg as urine enters and exists said cavity. In some preferred embodiments, the end portions are not corrugated. In another preferred embodiment, the reservoir includes a cushion extending along the lower longitudinal seam.

In another preferred embodiment, the urine reservoir preferably includes inner and outer overlapping, elongated strips of flexible, water-impermeable material formed into a double-wall, endless sleeve. An internal urine cavity is formed inside the walls of the sleeve. The sleeve envelops the user's lower leg and rests on the user's angle.

The systems optionally include a pressure sensor fixed to the outer surface of the inner strip in the central portion, or attached directly to the leg. Alternatively, the pressure sensor may be attached directly to the user's leg by a medical-grade glue or past and resides in a statis position. The sensor is made from rigid and flexible material and is attached to the 342/542 in a way that it's pressed to the leg and becomes shaped and contoured as a curved shape of the individual's particular and unique shape of his or her leg and the, vertically expanded conically-shaped corrugated reservoir keeps it intact.

### BRIEF DESCRIPTION OF THE DRAWINGS

FIG. 1 is a broken-away, part-sectional, reduced-scale, side view of one embodiment of some components of a catheter system in accordance with this disclosure.

FIG. 2 is an enlarged sectional view taken on line 2-2 of FIG. 1.

FIG. 2A is an enlarged sectional view of a modified detail taken on line 2A-2A of FIG. 2.

FIG. 2B is an enlarged sectional view of a modified detail taken on line 2B-2B of FIG. 2.

FIG. 3A is an enlarged sectional view taken on line 3A-3A of FIG. 1.

FIG. 3B is an enlarged sectional view taken on line 3B-3B of FIG. 13.

FIG. 4 is an enlarged, part-sectional, side view of additional components of the system of FIG. 1 depicting its mounting on a person's leg.

FIG. 5 is a top plan view of the components of FIG. 4 in accordance with one embodiment.

FIG. 6 is a broken-away, front elevation view of the components of FIG. 4 mounted on the person's leg.

FIG. 6A is a broken-away, side view of a modified detail of the system.

FIG. 6B is a broken-away, side view of a modified detail of the system.

FIG. 6C is a broken-away, side view of a modified detail of FIG. 6B.

FIG. 7 is a sectional view of a modified detail of the system.

FIG. 8 is an enlarged bottom plan view of another detail of the system.

FIG. 9 is an enlarged sectional view of another detail of the system.

FIG. 10 is a side view of another modified detail of the system.

FIG. 11 is a broken-away, enlarged side view of a further modified detail of the system.

FIG. 12 is a broken-away, sectional view of the detail of FIG. 11.

FIG. 13 is a broken-away, part-sectional, reduced-scale, side view of another embodiment of some components of a catheter system in accordance with this disclosure;

FIG. 14 is a side elevation of a system in accordance with another embodiment of the invention shown installed on a user's boot;

FIG. 15 is a sectional view taken along line 15-15 of FIG. 14;

FIG. 16 is a sectional view taken along line 16-16 of FIG. 14;

FIG. 17 is a sectional view of an alternative embodiment of the catch shown in FIG. 15;

FIG. 18 is a fragmented side elevation a urine reservoir in an uninstalled configuration in accordance with another embodiment of the invention;

FIG. 19 is top plan view of the urine reservoir of FIG. 19 in an installed configuration;

FIG. 20a is a fragmented, cross-section taken along line 20a-20a of FIG. 19 when the reservoir is empty;

FIG. 20b is a fragmented, cross-section taken along line 20b-20b of FIG. 19 when the reservoir is filled with urine;

FIG. 21 is a partial cross-section, partial side elevation of the urine reservoir of FIG. 18 installed on a human leg and partially filled with urine;

FIG. 22 is a fragmented side elevation a urine reservoir in an uninstalled configuration in accordance with another embodiment of the invention; and,

FIG. 23 is a partial cross-section, partial side elevation of a urine reservoir installed on a human leg and partially filled with urine in accordance with yet an additional embodiment of the invention.

### DETAILED DESCRIPTION OF PREFERRED EMBODIMENTS

Reference will now be made in detail to exemplary aspects of the present disclosure that are illustrated in the accompanying drawings. Wherever possible, the same reference numbers will be used throughout the drawings to refer to the same or like parts.

As used herein, the term “ankle” shall mean the bones that make up the angle joint, which include: the medial malleolus of the tibia, the lateral malleolus of the fibula, and the talus. The medial malleolus presents as a bulge on the lower inside portion of the leg, while the lateral malleolus presents as a bulge on the lower, outside portion of the leg.

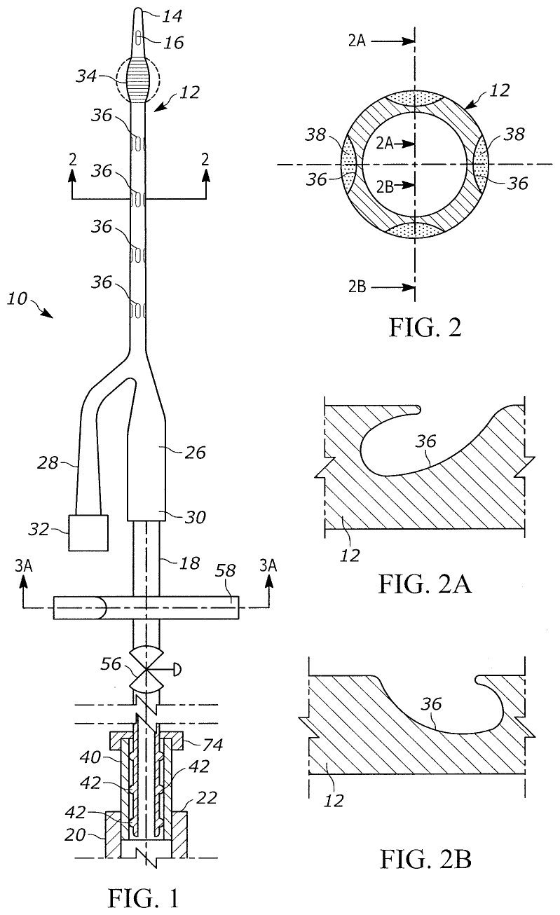

Referring now to FIGS. 1-6 of the drawings, one embodiment of a catheter system 10 comprises an indwelling, hollow catheter 12, typically constituted of a silicone material, or a coated natural latex material, or any medical grade material, having a generally cylindrical tip 14 inserted through a person's urethra and into the person's bladder in use, and at least two main and auxiliary, generally cylindrical, separate lumens 26, 28 having interior channels. The tip 14 has an opening 16 through which urine from the bladder passes through the urethra and into the main lumen 26 of the catheter 12 to a urine drainage port 30. The catheter 12 may have an optional, expandable balloon 34 adjacent the tip 14. The auxiliary lumen 28 may also have a fill port 32 through which a fluid, typically sterile water, is admitted to fill and expand the balloon 34 to its illustrated dashed line shape in FIG. 1 in order to anchor the tip 14 in the person's body. The fill port 32 also prevents backflow of the sterile water and an unauthorized collapse of the balloon 34.

The presence of the balloon 34 has sometimes caused a small amount of residual urine, e.g., 10-100 mL, to remain in the bladder. This residual urine can become infected, thereby sometimes resulting in chronic infection and scarring of the urethra and the bladder, as well as an encrustation to form around and within the catheter 12. This can block the opening 16 and the urine flow along the catheter 12, and may result in a risk of bladder spasm, as well as in a painful medical emergency that requires a rapid medical response. When the sterile water is eventually removed from the balloon 34 with a syringe inserted through the fill port 32 prior to catheter withdrawal, the balloon 34 may sometimes fail to collapse completely. This can result in a small rim that can make it difficult or even impossible to withdraw the catheter. In addition, the balloon 34 can sometimes break during insertion and be fragmented. Also, the balloon 34 can sometimes be expanded before complete insertion, thereby resulting in bleeding, bodily damage, and even rupture of the urethra. In long term usage, this can lead to permanent scarring of the urethral passage, for both men and women. In a preferred embodiment, the balloon 34 and auxiliary lumen 28 are eliminated.

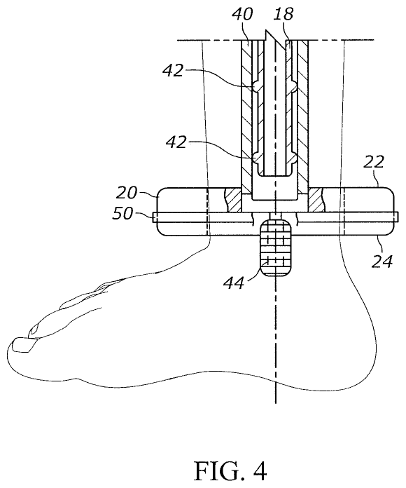

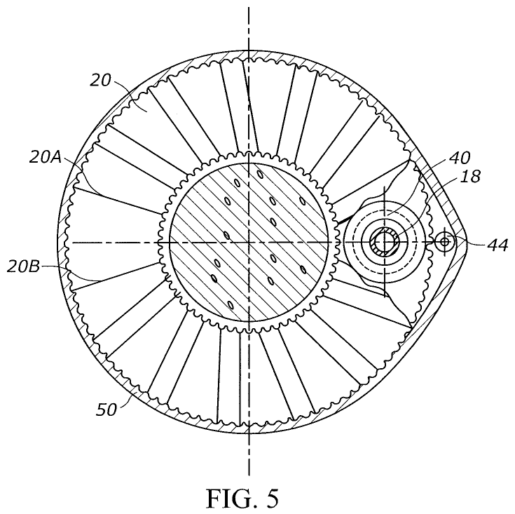

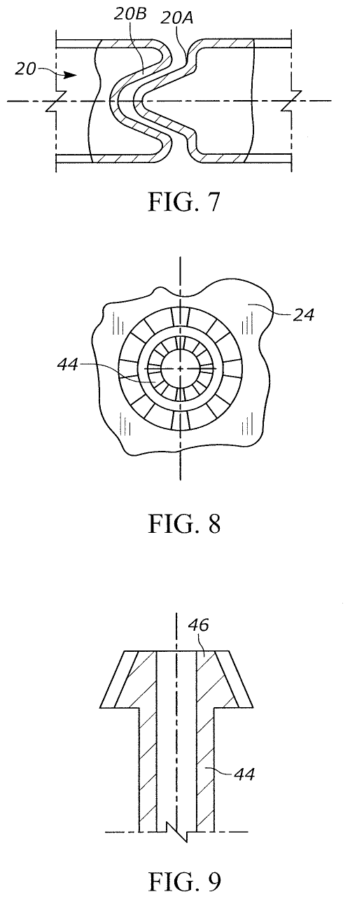

A flexible, hollow bag or reservoir 20 (see FIG. 4), typically constituted of a plastic material and configured as a cylinder, is advantageously bendable from an initial linear shape to a final curved, generally annular shape. Alternatively, the reservoir 20 may already be pre-formed into the curved annular shape. In use, the reservoir 20 extends circumferentially at least partially around the person's ankle and has walls bounding an interior compartment. As shown in FIG. 5, the curved reservoir 20 has opposite ends 20A, 20B that are spaced apart in the final shape. As shown in FIG. 7, the bent reservoir 20 has opposite ends 20A, 20B that engage each other, and may even interconnect with each other, for example, with a press fit or a snap fit, in order to keep the final shape around the ankle. As shown in FIG. 4, the walls of the reservoir may be smooth, or may be corrugated to facilitate the bending into the curved shape as shown in FIG. 5. The corrugated walls permit the reservoir 20 to be stretched and collapsed, as needed, during installation on the person.

A flexible, elongated, cylindrical tube 18, also typically constituted of a plastic medical grade material, has one end press-fitted to the urine drainage port 30 of the catheter 12, and an opposite end connected, as described below, to, and in fluid communication with, the reservoir 20. The tube 18 is hollow for conducting urine in the catheter 12 through the tube 18 into the interior compartment of the reservoir 20. The length of the tube 18 may be adjusted to fit different persons whose legs are of different lengths by cutting the tube 18 to a desired length. One of the walls of the reservoir 20 is a top wall 22, and another of the walls of the reservoir 20 is a bottom wall 24 that engages and rests on a top of the person's foot in use to resist downward movement of the reservoir 20 as the reservoir is filled with urine.

Advantageously, the catheter 12 has at least one pocket, and preferably a plurality of pockets 36, each filled with a lubricant 38 (see FIG. 2) to ease insertion and decrease friction of the catheter 12 through the urethra and into the bladder. As shown in FIG. 1, the pockets 36 may be spaced apart lengthwise along an axis of the catheter 12 and/or circumferentially around the catheter 12. As shown, each pocket 36 is a crater, preferably having a parabolic shape, for holding a maximum amount of the lubricant 38, which is beneficial for long term usage and to decrease friction between the catheter 12 and the urethral passage. It will be understood that any other shape for the pocket 36 may be employed. For example, see FIGS. 2A, 2B, wherein the lubricant pockets 36 are asymmetrical and oriented at different opposite angles of inclination relative to the axis to facilitate long term retention of the lubricant 38 in the pockets 36. In addition, a surface coating, such as a polytetrafluoroethylene, a hydrogel, or a silicon elastomer, may be applied over the exterior of the catheter 12, again to ease insertion and decrease friction.

A rigid, tubular, hollow, cylindrical sleeve 40, which is elongated along a longitudinal sleeve axis, is inserted into, and extends through, the top wall 22 into the compartment of the reservoir 20, and is securely held therein, preferably with a press fit or a snap fit. The opposite end of the tube 18 is cut, as described above, to accommodate and fit users having legs of different lengths. The opposite cut end of the tube 18 may be inserted into an upper end of the sleeve 40, and/or through a sealing cap 74, and is free to move and slide along the longitudinal sleeve axis as the person moves his legs and body, e.g., during his daily activities. The cap 74 serves to seal the interior of the sleeve 40.

Advantageously, the opposite end of the tube 18 has at least one annular barrel or slide collar 42, and preferably a plurality of annular barrels or slide collars 42 that are spaced apart along the longitudinal sleeve axis. The collars 42 make a sliding, sealing contact with the interior wall of the sleeve 40 and serve as an automatic length compensator to accommodate relative movement between the tube 18 and the sleeve 40 during the person's movement. The collars 42 also serve to reduce friction between the tube 18 and the sleeve 40 during their relative movement.

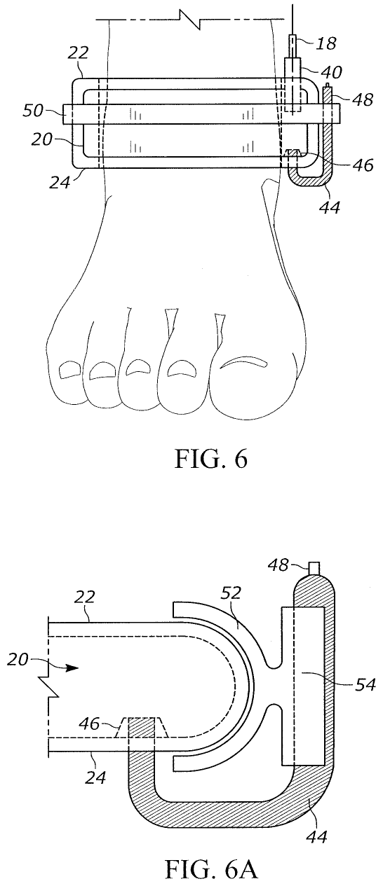

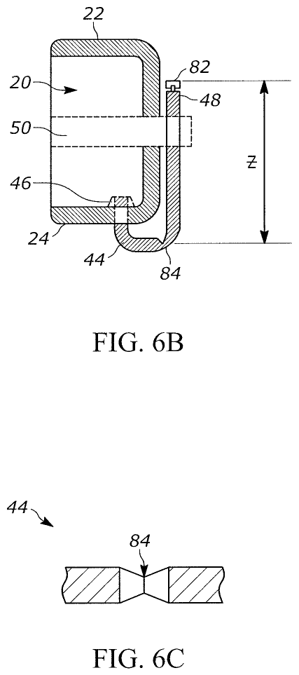

A flexible, tubular, hollow, drain pipe 44 (see FIG. 6), preferably constituted of a corrugated plastic material, extends between an inlet pipe end 46 and its opposite outlet pipe end 48. The inlet pipe end 46 is inserted into, and extends through, the bottom wall 24 into the compartment of the reservoir 20, and is securely held therein, preferably with a press fit or a snap fit. As shown in FIG. 9, the inlet pipe end 46 preferably has a frustoconical shape. As shown in FIG. 8, the bottom wall 24 has an opening in which the inlet pipe end 46 is received, and radial slits bound the opening so as to closely engage and fluid-tightly seal the pipe end 46 and prevent its accidental disconnection.

The drain pipe 44 is flexed and manipulated in any direction in use so as to move the outlet pipe end 48 relative to the inlet pipe end 46 between a raised position above the bottom wall 24 as shown in FIG. 6, and a lowered position in which the outlet pipe end 48 is positioned below the bottom wall 24 in order to empty the compartment of the reservoir 20 of urine. The corrugated drain pipe 44 may be stretched (expanded) and collapsed (retracted), as needed, during movement between the raised and lowered positions.

An elastic, endless band 50 surrounds the outlet pipe end 48 to hold the outlet pipe end 48 in its raised position as the stretched elastic band 50 seeks to return to its original shape. Alternatively, the band 50 may be a belt having hook-and-loop fasteners. When it is desired to empty the reservoir 20, the band 50 is moved to expose and release the outlet pipe end 48 for manipulation and movement. Similarly, the band 50 also surrounds the reservoir 20 to hold the reservoir 20 in its final shape and in its mounted position atop the person's foot. In a variant construction, as shown in FIG. 6A, a bracket 52 may be positioned and held at a side of the reservoir 20. The bracket 52 has a pair of side arms 54 bounding a U-shaped channel in which the drain pipe 44 may be snapped in place and secured in the raised position, as well as being readily unsnapped in order to move the outlet pipe end 48 to the lowered position.

As best shown in FIG. 6B, the outlet pipe end 48 of the drain pipe 44 may be closed with a removable cap 82. In addition, the drain pipe 44 may advantageously be formed with a bending line 84 intermediate its ends 46, 48. In the raised position shown in FIG. 6B, the drain pipe 44 is kinked at the bending line 84, thereby shutting off any urine flow towards the outlet pipe end 48. When the outlet pipe end 48 is lowered by being moved over the distance “Z” in FIG. 6B, then the drain pipe 44 is unkinked, as shown in FIG. 6C, in which case, urine is free to flow towards the outlet pipe end 48.

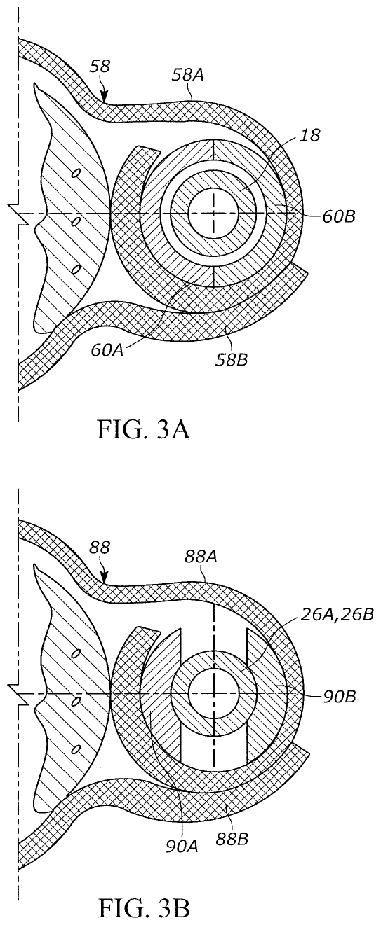

Returning to FIG. 1, a manual valve 56 may be positioned anywhere along the tube 18, typically positioned below the person's knee, to control the urine flow. Also, a guide strap 58 encircles the person's leg, and is typically positioned below the person's knee. As best shown in FIG. 3A, the guide strap 58 has two opposite strap ends 58A, 58B having mutually contacting U-shaped members 60A, 60B between which the tube 18 is received with mechanical clearance, thereby allowing the tube 18 to have freedom of movement and to be freely guided when the strap ends 58A, 58B are fastened together, for example, with hook-and-loop fasteners, such as Velcro (trademark).

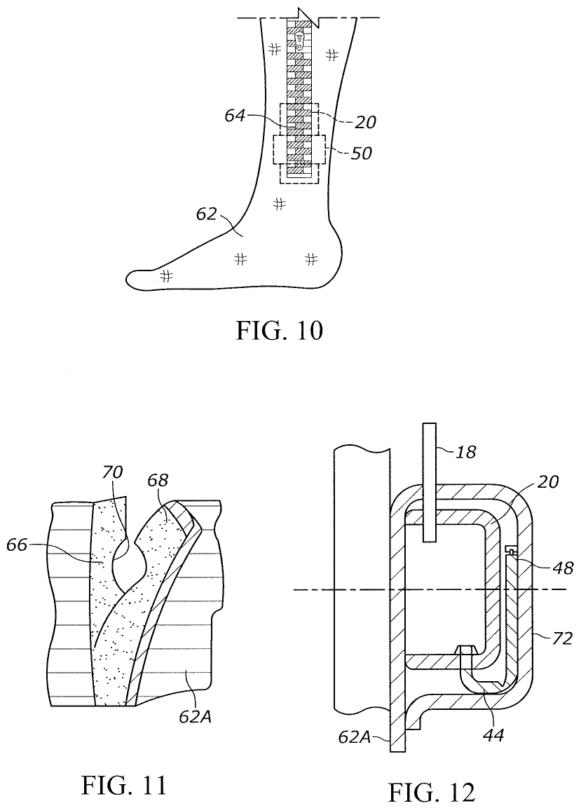

Instead of the endless band 50, a stocking 62 (see FIG. 10) may be worn over the reservoir 20 and the drain pipe 44, in order to enclose and hold the reservoir 20 in its final shape and in its mounted position atop the foot and around the ankle, as well as to hold the outlet pipe end 48 in its raised position. The stocking 62 has an openable fastener, such as a zipper 64 that, when opened, allows ready access to the outlet pipe end 48 of the drain pipe 44.

Instead of the zipper 64, the fastener may comprise a pair of hook-and-loop fasteners 66, 68, such as Velcro (trademark), as shown in FIGS. 11-12, on neck portions of a stocking 62A that is worn under the reservoir 20 to prevent skin irritation from the reservoir 20. The neck portions form an opening 70 therebetween. The tube 18 is received in the opening 70 in use. The upper annular neck of the stocking 62A is folded over to form an annular cuff 72 that surrounds and holds the outlet pipe end 48 in its raised position, and also surrounds and holds the reservoir 20 in its final shape and in its mounted position atop the foot and around the ankle.

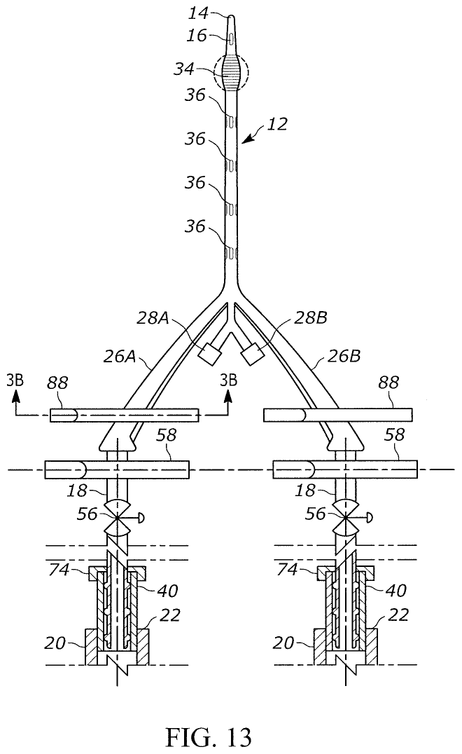

FIG. 13 depicts another embodiment of a urine collection and drainage system that is analogous to that shown in FIG. 1, and hence, like reference numerals have been used to identify like parts. Instead of one main lumen 26 and one auxiliary lumen 28, the embodiment of FIG. 13 depicts two main lumens 26A, 26B and two auxiliary lumens 28A, 28B. The main lumens 26A, 26B are routed to the person's right and left legs. This allows any desired distribution, for example, an equal distribution, of urine to the right and left legs to maximize the capacity of the urine being collected. Valves 56 on the main lumens 26A, 26B may be operated to redirect the urine flow as needed to the reservoir on each leg, as well as in the event of malfunction of the main lumens 26A, 26B. One of the auxiliary lumens 28A, 28B may be used to expand the balloon 34, while the other auxiliary lumen may be used to inject medications.

As also shown in FIG. 13, a holding strap 88 encircles the person's leg, and is typically positioned above each person's knee. As shown in FIG. 3B, each holding strap 88 has two opposite strap ends 88A, 88B having a pair of U-shaped members 90A, 90B that are spaced apart and that clamp each main lumen 26A, 26B therebetween in a fixed position when the strap ends 88A, 88B are fastened together, for example, with hook-and-loop fasteners, such as Velcro (trademark). The holding straps 88 are used to help anchor the main lumens 26A, 26B on the person's leg, in order to prevent the catheter 12 from being pulled down.

As previously mentioned, the balloon 34, which is known in the art, is preferably eliminated to avoid the aforementioned problems. The resting of the reservoir 20 atop the person's foot, the holding straps 88, and the compensating relative movement between the tube 18 and the sleeve 40 all serve to prevent the urine-filled weighted reservoir 20 from pulling the catheter 12 down.

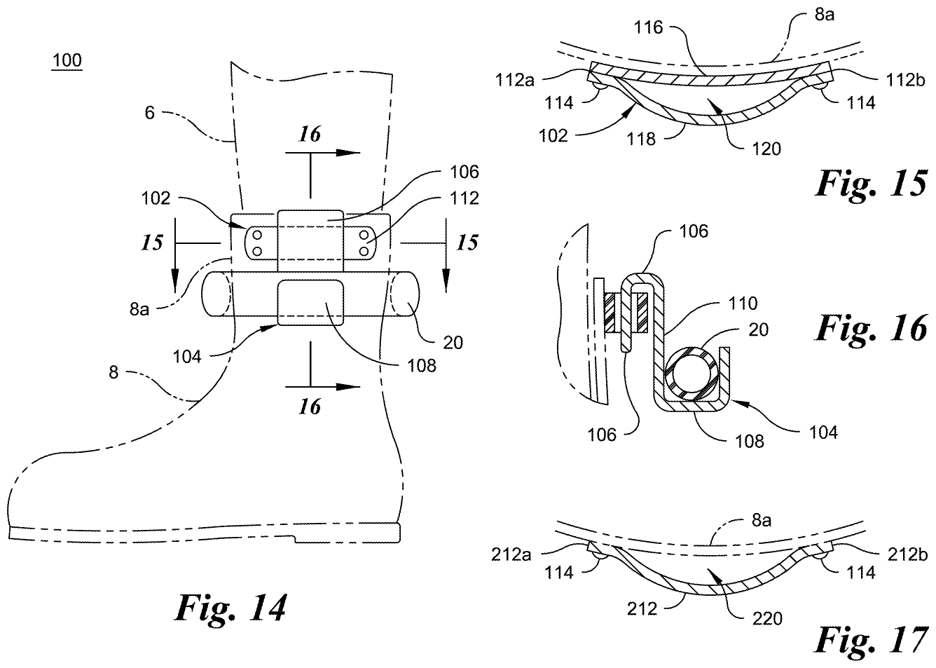

An apparatus in accordance with an additional embodiment of the invention is shown in FIGS. 14-17. In this embodiment of the invention, the reservoir 20 is not strapped to or supported by the catheter user's leg or ankle; instead, the reservoir 20 is supported by an external article of footwear or clothing, such as the user's shoe, boot, pant leg, other garment, etc., or other structure. Although described below with respect to a boot, it should be appreciated by one of ordinary skill in the art that the apparatus would work the same way with the other aforementioned external fixation locations.

Referring to FIGS. 14-16, the apparatus 100 in accordance with this embodiment is shown installed on a boot 8, which is worn by the catheter user. The apparatus 100 generally comprises a catch, designated generally by reference numeral 102, which is fixed to the upper 8a of the boot 8, and a carrier, designated generally by reference numeral 104, which supports a urine reservoir 20 above the user's foot by removable affixation to the catch 102. In this embodiment, the carrier 104 may support a urine reservoir 20 having the shape and construction described above with reference to FIGS. 1-13.

In the preferred embodiment shown in FIGS. 14-16, the carrier 104 comprises an S-shaped strip of rigid material, such as metal or plastic, having a mounting-hook portion 106 and a U-shaped cradle portion 108 conjoined by a central shank portion 110 as best seen in FIG. 16. The cradle portion 108 is shaped and dimensioned so that at least a portion of the urine reservoir 20 is seated in and supported by the cradle portion 124. Referring to FIG. 14, in this embodiment, the urine reservoir 20 surrounds the user's lower leg in the manner described above with respect to the embodiments shown in FIGS. 1-13.

In the preferred embodiment shown in FIGS. 14-16, the catch 102 comprises a strip 112 of durable material, such as leather or plastic, which is fastened at opposed ends 112a, 112b to the upper 8a of the boot 8 using rivets 114. In other preferred embodiments, the strip 112 may be fastened to the upper 8a of the boot 8 using stitches, pins, Velcro, snaps, adhesives or other mechanical fasteners. The strip 112 is made from a material that is preferably flexible enough to conform to the natural shape of the boot upper 8a, but durable enough to support the carrier 104. To maintain the aesthetic appearance of the boot 8, the strip is preferably made from the same material as the boot upper 8a or another material that blends with the appearance of the boot so that the boot 8 can also be worn when the user is not wearing a catheter.

In a preferred embodiment, the catch 102 comprises a strip 112 of durable, semi-flexible material having a two-ply construction comprising a first, inner layer 116 and a second, outer layer 118. In one preferred embodiment, the layers 116, 118 are overlapping and/or partially adhered to one another at opposed ends, but are unadhered in the middle and form a blind or open-ended pocket 120 intermediate the layers 116, 118 and intermediate the points of affixation to the boot upper 8a as best seen in FIG. 15. Preferably, the pocket 120 has a size and shape that closely compliments the size and shape of the hook portion 106 of the carrier 104 so that the hook portion 106 fits snugly but removably within the pocket 120.

In another preferred embodiment, the carrier 102 comprises a strip 212 of durable, semi-flexible material having a single-ply construction, which is fastened at opposed ends 212a, 212b to the upper 8a of the boot 8 using rivets 114 or other mechanical fasteners or adhesives such as described above. Because the strip 212 and boot 8 are flexible, the hook portion 106 can be inserted into the gap 220 between strip 212 and boot upper 8a by simply deflecting either material in a direction opposite the other material.

In a similar manner, the catch 102 can be affixed to the outside of the user's shoe, pant leg or other garment. One of ordinary skill in the art could readily adjust the axial location (along the length of the user's leg) of the urine reservoir by changing the position at which the catch 102 is affixed to the boot upper, shoe or pant leg, as well as changing the length of the shank portion 110 of the carrier 102.

In this embodiment, the location of the catch 102 is preferably selected so that the cradle portion 108 of the carrier 102 sits above either the medial or lateral portion of the user's ankle. When a single apparatus 100 is used, a large portion of the weight of the urine reservoir 20 is supported by the carrier 104 above the ankle, while the remaining portion of the reservoir 20 diametrically opposed to the carrier 104 rests against the user's leg and/or on the opposed portion of the ankle. Therefore, in preferred embodiments, multiple catch/carrier combinations are spaced around the periphery of the boot 8 so that the urine reservoir 20 is fully supported at those multiple locations, which transfers all of the weight of the reservoir 20 to the boot 8. The apparatus preferably includes at least two catch/carrier combinations, more preferably three catch/carrier combinations.

The apparatus 100 of this embodiment of the invention holds the urinary reservoir 20 stable and in a fixed position during a variety of everyday environments and activities. However, the reservoir 20 and carrier 104 can be easily removed from the boot 8 and supported on an external structure when desired. For example, the carrier 104 and reservoir 20 may be removed from the catch 102 and supported at a location on a bed frame while sleeping so long as the attachment location enables the bag to be filled by the force of gravity.

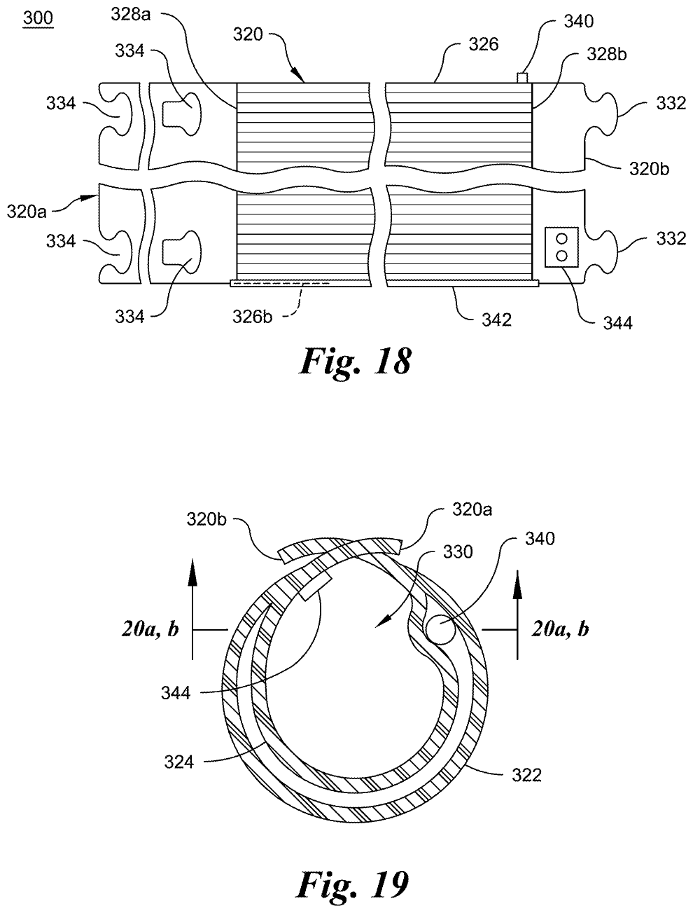

A urinary catheter system 300 in accordance with yet a further embodiment of the invention is shown in FIGS. 18-21. In this embodiment of the invention, the reservoir 320 is not strapped to or supported by the catheter user's upper leg. Instead, the reservoir 320 surrounds the user's lower leg and is supported on the user's ankle in a manner similar to the embodiments described above and shown in FIGS. 1-13. The system 300 of this embodiment of the invention holds the urinary reservoir 320 in a fixed position in a variety of everyday environments and activities. The reservoir 320 does not slide downwardly below the ankle because the inner diameter of the reservoir 320, once installed, is smaller than the maximum diameter of the ankle at the medial and lateral malleoli. Moreover, the reservoir 320 does not slide upwardly because the diameter of the human leg generally increases along the lower leg moving proximally from the ankle toward the calf, while the inner diameter of the reservoir 320 remains constant or is reduced due to expansion as urine fills the reservoir.

Referring to FIGS. 18-21, the system 300 in accordance with one preferred embodiment generally comprises a novel urine reservoir, designated generally by reference numeral 320, connected via tubing to a urinary catheter in the same manner as described above with reference to FIGS. 1-13. In one preferred embodiment, the urine reservoir 320 is formed from overlapping inner 324 and outer 322 strips of flexible, material that is impermeable to water. With reference to FIG. 18, an internal urine cavity 330 is formed by sealing the strips together along a periphery defined by upper 326a and lower 326b longitudinal seams, and left 328a and right 328b lateral seams.

Similar to the embodiment described in FIGS. 1-13, the reservoir 320 has a rigid, cylindrical tube 340, inserted into and extending through the top portion of the urine cavity 330, and is securely held therein, preferably with a press fit or a snap fit. The opposite end of the tube 340 connects to tubing, such as tubing 18 in FIG. 1, in fluid communication with a urinary catheter, such as catheter 12 in FIG. 1. The tube 340 is cut to accommodate and fit users having legs of different leg lengths.

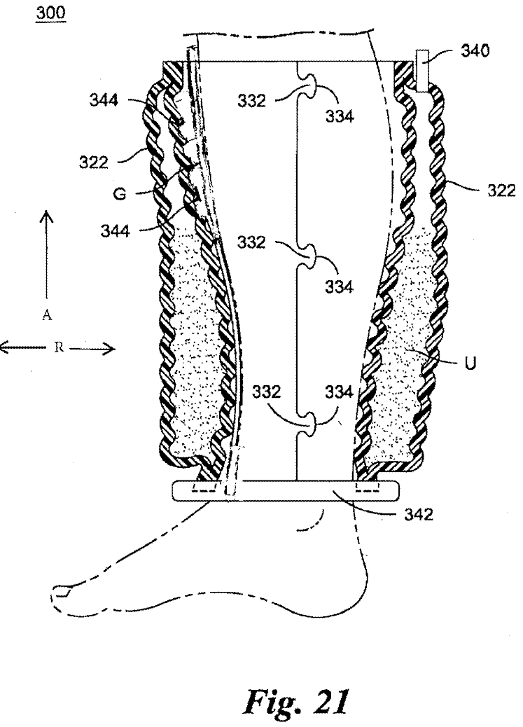

To releaseably secure the reservoir 320 around on around the periphery of the user's lower leg, connection means are formed on opposed ends 320a, 320b of the reservoir 320. Referring to FIG. 18, in one preferred embodiment the connection means comprises a plurality of tabs 332 formed at one end 320b, which engage complimentary-shaped and sized slits 334 formed in the other end 320a of the reservoir 320. In a preferred embodiment, the reservoir 320 includes two or more sets of slits 334 arranged at different longitudinal locations on one end 320a, such as show in FIG. 18, so that the reservoir can be closed snugly around different-sized (diameter) legs. Once the ends are connected, such as shown in FIGS. 19 and 21, the reservoir 320 removably encircles the user's lower leg and rests on the medial and lateral malleoli. In one preferred embodiment, the reservoir includes a cushion 342 on the lower lateral seam 326b, which is the portion that will rest on the ankle when the reservoir is filled.

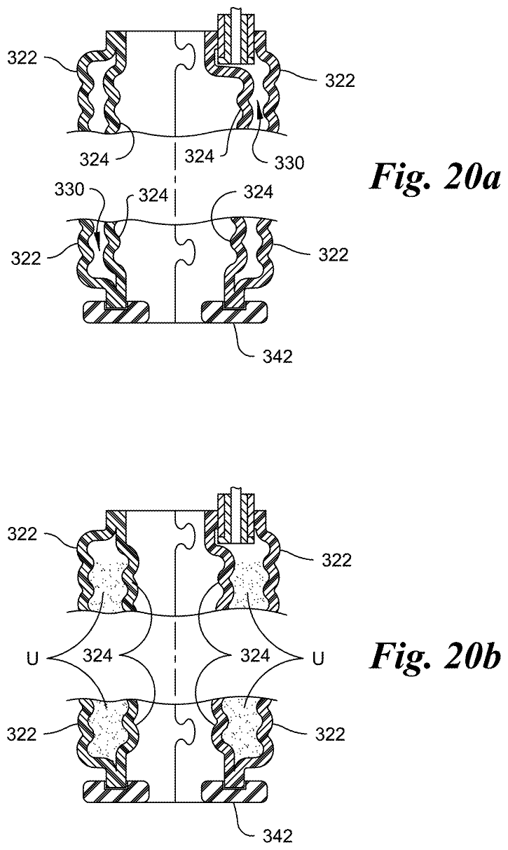

In a preferred embodiment, the central portion of the reservoir 320 is corrugated or pleated (depicted by solid horizontal lines in FIG. 18), which allows the reservoir to be stored/shipped and installed in a collapsed configuration, and then to grow/expand laterally as the reservoir is filled with urine “U”, depicted by liquid stippling in FIG. 20b. When the reservoir 320 is initially installed around the user's ankle, it fits loosely around the lower leg and rests on the medial and lateral malleoli of the ankle. A gap “G” is formed between the reservoir and the leg. As it is filled with urine, the reservoir 320 expands radially relative to the user's tibia, shown by direction arrow “R” in FIG. 21, but also expands axially upwardly relative to the user's tibia, shown by direction arrow “A” in FIG. 21 toward the calf due to the lengthwise-extending corrugations/pleats in the central portion of the reservoir 320.

As the reservoir 320 expands, the internal diameter decreases and begins to apply pressure on the user's lower leg. Once the pressure reaches an observable limit, experienced users know the reservoir is full; however, first-time or inexperienced users may not know when the bag is full without visual observation. Therefore, the system may be provided with a pressure sensor 344 located on the outer surface of the inner strip 324, which contacts the leg during expansion. The pressure sensor 344 preferably communicates with the user's smart phone by sending an alert advising the user that the reservoir 320 is full or almost full.

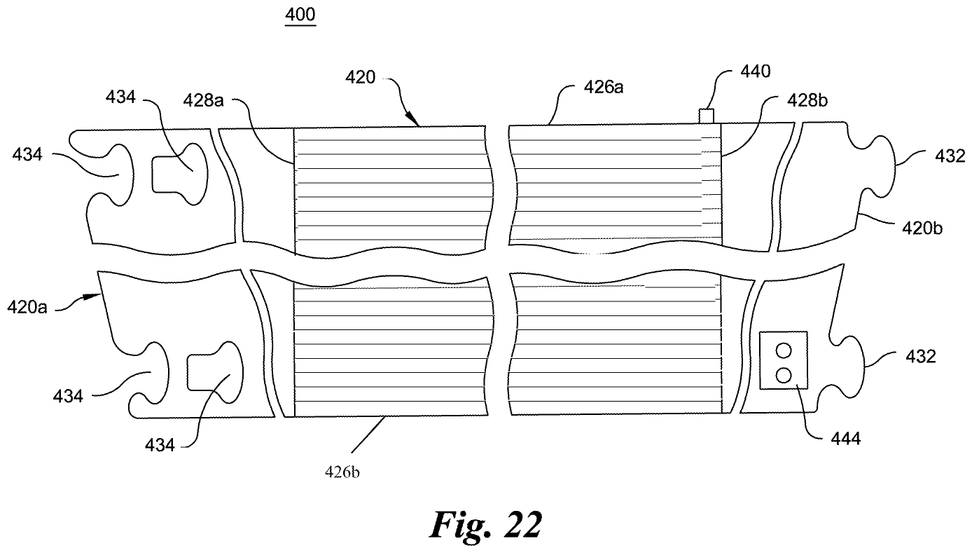

A urinary catheter system 400 in accordance with yet a further embodiment of the invention is shown in FIG. 22. In this embodiment of the invention, the reservoir 420 is not strapped to or supported by the catheter user's upper leg. Instead, the reservoir 420 surrounds the user's lower leg and is supported on the user's ankle in a manner similar to the embodiments described above and shown in FIGS. 1-13 and FIGS. 18-21.

Referring to FIG. 22, the system 400 in accordance with this preferred embodiment generally comprises a novel urine reservoir, designated generally by reference numeral 420, connected via tubing to a urinary catheter in the same manner as described above with reference to FIGS. 1-13. In this preferred embodiment, the urine reservoir 420 is formed from overlapping inner 424 and outer 422 strips of flexible, material that is impermeable to water. With reference to FIG. 22, an internal urine cavity 420 is formed by sealing the strips together along a periphery defined by upper 426a and lower 426b longitudinal seams, and left 428a and right 428b lateral seams.

The system has essentially the same construction, and functions essentially the same way, as the system 300 described above with reference to FIGS. 18-21. However, in this embodiment, the length of the strips of flexile material 422, 424 is greater along the upper longitudinal seam 426a than the lower longitudinal seam 426b. The length tapers outwardly proceeding from the bottom to the top as referenced in FIG. 22, which shape compliments the tapered shape of the average human lower leg.

Similar to the embodiment described in FIGS. 1-13 and 18-21, the reservoir 420 has a rigid, cylindrical tube 440, inserted into and extending through the top portion of the urine cavity 420, and is securely held therein, preferably with a press fit or a snap fit. The opposite end of the tube 440 connects to tubing, such as tubing 18 in FIG. 1, in fluid communication with a urinary catheter, such as catheter 12 in FIG. 1. The tube 440 is cut to accommodate and fit users having legs of different leg lengths.

The reservoir is secured around the periphery of the user's lower leg in the same manner as the system 300 described above using the connection means formed on opposed ends 420a, 420b of the reservoir 420. Referring to FIG. 22, in this preferred embodiment the connection means comprises a plurality of tabs 432 formed at one end 420b, which engage complimentary-shaped and sized slits 434 formed in the other end 420a of the reservoir 420. The reservoir 420 includes two or more sets of slits 434 arranged at different longitudinal locations on one end 420a, such as show in FIG. 22, so that the reservoir can be closed snugly around different-sized (diameter) legs. In other preferred embodiments, the reservoir may be connected with Velcro, snaps, or other mechanical fasteners. Once the ends are connected, the reservoir 420 removably encircles the user's lower leg and rests on the medial and lateral malleoli in a manner similar to the embodiment shown in FIGS. 21.

In a preferred embodiment, the central portion of the reservoir 420 is corrugated or pleated (depicted by solid horizontal lines in FIG. 22), which allows the reservoir to be stored/shipped and installed in a collapsed configuration, and then to grow/expand laterally as the reservoir is filled with urine in the same manner as described above with respect to the system shown in FIG. 21. When the reservoir 420 is initially installed around the user's ankle, it fits loosely around the lower leg and rests on the medial and lateral malleoli of the ankle. As it is filled with urine, the reservoir 420 expands slightly outwardly, but also expands laterally/upwardly toward the calf due to the lengthwise-extending corrugations/pleats in the central portion of the reservoir 420 in the same manner as shown in FIGS. 18-21.

The system may be provided with a pressure sensor 444 located on the outer surface of the inner strip 424, which contacts the leg during expansion. The pressure sensor 444 preferably communicates with the user's smart phone by sending an alert advising the user that the reservoir 420 is full or almost full.

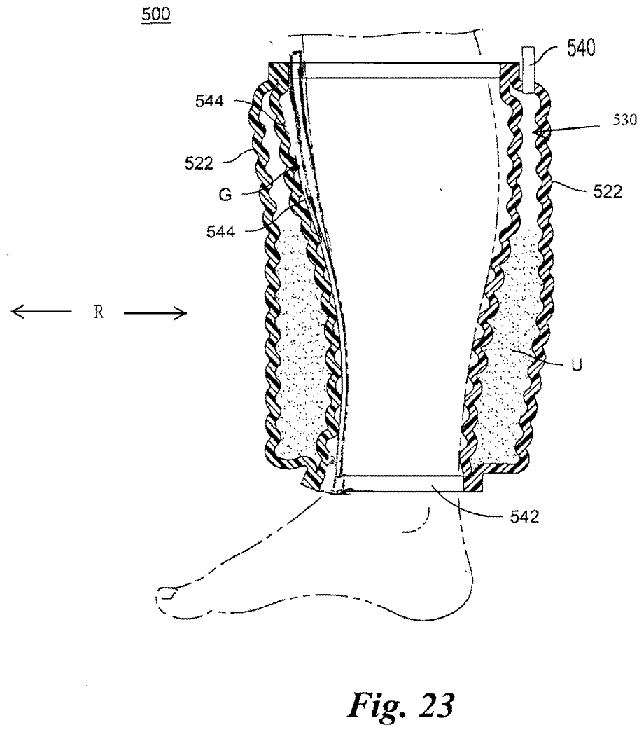

A urinary catheter system 500 in accordance with yet a further embodiment of the invention is shown in FIG. 23. Similar to the embodiments shown in FIGS. 16-22, the reservoir 520 is not strapped to or supported by the catheter user's upper leg. Instead, the reservoir 520 surrounds the user's lower leg and is supported on the user's ankle. The system 500 of this embodiment of the invention holds the urinary reservoir 520 in a fixed position in a variety of everyday environments and activities. The reservoir 520 does not slide downwardly below the ankle because the inner diameter of the reservoir 520, once installed, is smaller than the maximum diameter of the ankle at the medial and lateral malleoli. Moreover, the reservoir 520 does not slide upwardly because the diameter of the human leg generally increases along the lower leg moving proximally from the ankle toward the calf, while the inner diameter of the reservoir 520 remains constant or is reduced due to expansion as urine fills the reservoir.

Referring to FIG. 23, the system 500 in accordance with one preferred embodiment generally comprises a novel urine reservoir, designated generally by reference numeral 520, connected via tubing to a urinary catheter in the same manner as described above with reference to FIGS. 1-13. In one preferred embodiment, the urine reservoir 520 is formed from overlapping inner 324 and outer 322 strips of flexible, material that is impermeable to water. With reference to FIG. 23, the internal urine cavity is formed by sealing the strips together along upper and lower longitudinal seams. However, unlike the embodiments shown in FIGS. 16-22, this cavity has an endless construction, which forms a sleeve (similar to the bottom portion of a pant leg) into which the user inserts his/her leg. The sleeve eliminates the need to connect the free ends of the embodiments shown in FIGS. 16-22. Once installed, the reservoir 520 removably encircles the user's lower leg and rests on the medial and lateral malleoli. In one preferred embodiment, the reservoir includes an elastic band 542 at the bottom to support the reservoir in a fixed position above the ankle.

Similar to the embodiment described in FIGS. 1-13, the reservoir 520 has a rigid, cylindrical tube 540, inserted into and extending through the top portion of the urine cavity 530, and is securely held therein, preferably with a press fit or a snap fit. The opposite end of the tube 540 connects to tubing, such as tubing 18 in FIG. 1, in fluid communication with a urinary catheter, such as catheter 12 in FIG. 1. The tube 540 is cut to accommodate and fit users having legs of different leg lengths.

Similar to the embodiments shown in FIGS. 16-22, the central portion of the reservoir 520 is corrugated or pleated (depicted by solid wavy lines in FIG. 23), which allows the reservoir to be stored/shipped and installed in a collapsed configuration, and then to grow/expand laterally as the reservoir is filled with urine “U”, depicted by liquid stippling in FIG. 23. When the reservoir 520 is initially installed around the user's ankle, it fits loosely around the lower leg and rests on the medial and lateral malleoli of the ankle. A gap “G” is formed between the reservoir and the leg. As it is filled with urine, the reservoir 520 expands radially relative to the user's tibia, shown by direction arrow “R” in FIG. 23.

As the reservoir 520 expands, the internal diameter decreases and begins to apply pressure on the user's lower leg. Therefore, the system may be provided with a pressure sensor 544 located on the outer surface of the inner strip 324, which contacts the leg during expansion. The pressure sensor 544 preferably communicates with the user's smart phone by sending an alert advising the user that the reservoir 520 is full or almost full.

In all of the above-described embodiments, the pressure sensor could also be attached directly to the user's leg using a medical grade adhesive. In these embodiments, the corrugated surface of the reservoir contacts the pressure sensor as the reservoir is filled with urine.

Having described the preferred aspects and implementations of the present disclosure, modifications and equivalents of the disclosed concepts may readily occur to one skilled in the art. However, it is intended that such modifications and equivalents be included within the scope of the claims which are appended hereto.

1. An apparatus for supporting a urinary catheter collection reservoir on an article of footwear or clothing of a catheter user, comprising:
a) a catch having means for fastening said catch to the article; and
b) a carrier including a hook portion configured to be received within and supported by said catch, and a cradle portion configured to receive and support at least a portion of said reservoir.

2. The apparatus recited in claim 1, wherein said catch comprises an elongate strip of material, and said fastening means are located at each end of said strip.

3. The apparatus recited in claim 2, wherein said catch includes a pocket having a shape and size that compliments the shape and size of the hook portion of said carrier.

4. The apparatus recited in claim 3, wherein said catch comprises a two-ply strip of material, wherein said pocket is formed between said plies of material.

5. The apparatus recited in claim 2, wherein said strip forms a pocket between said article and said strip when fastened to the article, said pocket having a size and shape that compliments the size and shape of the hook portion of said carrier.

6. The apparatus recited in claim 2, wherein said fastening means comprises a mechanical fasteners selected from the group consisting essentially of rivets, pins, Velcro tabs, and snaps.

7. The apparatus recited in claim 2, wherein said fastening means comprises an adhesive.

8. The apparatus recited in claim 1, wherein said carrier comprises an S-shaped strip of rigid material, having a mounting-hook portion, and a U-shaped cradle portion conjoined by a central shank portion.

9. The apparatus recited in claim 1, wherein the catch and carrier are configured and sized so that the entire weight of the reservoir is supported on the article and not on the user's foot or ankle.

10. A urinary catheter system comprising:
a) an indwelling, catheter having a tip inserted into a person's bladder, said tip having an opening through which urine from the bladder flows into the catheter,
b) a urine reservoir constructed to wrap around and attach to a catheter user's lower leg above the ankle, comprising
i) inner and outer overlapping, elongated strips of flexible, water-impermeable material having a central portion and opposed end portions; said strips having a longitudinal length and a transverse width;
ii) an internal urine cavity formed in the central portion between said strips by a sealed periphery comprising upper and lower longitudinal seams and left and right lateral seams;
iii) first and second inter-engageable fasteners formed at opposed, lengthwise ends of said strips;
c) a flexible, elongated tube having one end connected to said catheter and an opposite end connected to said urine reservoir.

11. The urinary catheter system recited in claim 10, wherein said urine reservoir has a length sufficient to connect end-to-end at said fasteners, and to completely surround a user's lower leg.

12. The urinary catheter system recited in claim 11, wherein said urine reservoir has multiple fasteners arranged at different lengthwise positions to change the connected circumference of said urine reservoir.

13. The urinary catheter system recited in claim 12, wherein said fasteners comprise male tabs formed at one end and complimentarily-shaped and sized female slits formed at the other end.

14. The urinary catheter system recited in claim 11, wherein the walls of said urine reservoir are corrugated in the central portion forming said urine cavity.

15. The urinary catheter system recited in claim 14, wherein the corrugated walls of said urine reservoir have lengthwise-extending pleats so that the reservoir can expand and contract in the widthwise direction as urine enters and exists said cavity.

16. The catheter system recited in claim 15, wherein the end portions are not corrugated.

17. The catheter system recited in claim 10, including a pressure sensor fixed to the outer surface of said inner strip in the central portion.

18. The catheter system recited in claim 10, including a cushion extending along the lower longitudinal seam.

19. A catheter system comprising:
a) an indwelling, catheter having a tip designed for insertion into a catheter user's bladder, said tip having an opening through which urine from the bladder passes into the catheter, said catheter including a catheter wall defining at least one pocket for the retention of lubricant including a pocket opening defined on a surface of the catheter wall and a cavity extending into the catheter wall, wherein a first portion of the pocket opening overhangs the cavity and a second portion of the pocket opening does not overhang the cavity to create an asymmetrical parabolic shaped cavity;
b) a pair of flexible reservoirs, each of which is constructed to extend around the catheter user's ankle and has walls bounding an interior compartment; and
c) flexible tubing connecting said catheter in fluid communication with each of said reservoirs to conduct urine from said catheter into the compartment of said reservoirs;
wherein said reservoirs rest on the top of each of the user's feet to resist downward movement of the reservoir as the reservoir is filled with urine.

20. A catheter system comprising:
a) an indwelling, catheter having a tip designed for insertion into a catheter user's bladder, said tip having an opening through which urine from the bladder passes into the catheter,
b) a flexible reservoir extending around the person's ankle and having walls bounding an interior compartment; and
c) a flexible, elongated tube having one end connected to the catheter and an opposite end connected to the reservoir, the tube being hollow for conducting urine in the catheter through the tube into the compartment of the reservoir;
wherein one of the walls of the reservoir is a bottom wall that engages and rests on a top of the person's foot in use to resist downward movement of the reservoir as the reservoir is filled with urine.

21. The system of claim 20, and a flexible drain pipe having an inlet end in fluid communication with the compartment of the reservoir, and an outlet end movable between a raised position above the bottom wall and a lowered position below the bottom wall.

22. The system of claim 21, and a stocking worn on the person's leg and surrounding and enclosing the reservoir to hold the reservoir against the person's leg atop the person's foot, and also enclosing the outlet end to hold the outlet end in the raised position, the stocking having an openable fastener.

23. The system of claim 21, and a stocking worn on the person's leg and having a foldable neck to form a cuff that surrounds and holds the reservoir between the cuff and the neck, and also enclosing the outlet end in the raised position, the stocking having an openable fastener.
